(19) United States
(12) Plant Patent Application Publication
(10) Pub. No.: US 2015/0230374 P1
(43) Pub. Date: Aug. 13, 2015

(54) STRAWBERRY PLANT NAMED 'FRONTERAS'

(50) ;

(71) Applicant: The Regents of the University of California, Oakland, CA (US)

(72) Inventors: Kirk D. Larson, Santa Ana, CA (US); Douglas V. Shaw, Davis, CA (US)

(73) Assignee: The Regents of the University of California, Oakland, CA (US)

(21) Appl. No.: 13/999,312

(22) Filed: Feb. 10, 2014

(51) Int. Cl. A01H 5/00

(52) U.S. Cl. PLT/208

(57) ABSTRACT

‘Fronteras’ is a short-day (June bearing) cultivar similar to ‘Camarosa’ (U.S. Plant Pat. No. 8,708), but with greater productivity, higher quality fruit, and earlier production; it is similar to ‘Ventana’ (U.S. Plant Pat. No. 13,469) and ‘Benicia’ (U.S. Plant Pat. No. 22,542), but with somewhat later production, a larger plant, superior fruit and quality, and better-flavored fruit.

### GENUS AND SPECIES

The strawberry cultivar of this invention is botanically identified as Fragaria×ananassa Duch.

### VARIETY DENOMINATION

The variety denomination is ‘Fronteras’.

### BACKGROUND OF THE INVENTION

This invention relates to a new and distinctive short-day type cultivar designated as ‘Fronteras’, which resulted from a cross performed in 2008 between two unreleased germplasm accessions Cal 4.18-4 and Cal 5.165-1. Accession Cal 4.18-4 was chosen as a parent due to its very high early productivity, large and high quality fruit, and moderate plant vigor. Accession Cal 5.165-1 was chosen as a parent due to its vigorous but open plant habit and firm, large and flavorful fruit, and extended productivity.

‘Fronteras’ was first fruited at the University of California South Coast Research and Extension Center, near Irvine, Calif. in 2009, where it was selected, originally designated Cal 8.132-608, and propagated asexually by runners. Following selection and during testing the plant of this selection was designated ‘C235’. With the decision that this plant was to be released, this plant was given the name ‘Fronteras’ for purposes of introduction into commerce and for international registration and recognition. Asexual propagules from this original source have been tested at the Watsonville Strawberry Research Facility, the South Coast Research and

Extension Center, and to a limited extent in grower fields starting in 2010. The cultivar is stable and reproduces true to type in successive generations of asexual reproduction.

### BRIEF SUMMARY OF THE INVENTION

‘Fronteras’ is a short-day (June bearing) cultivar similar to ‘Camarosa’ (U.S. Plant Pat. No. 8,708), but with greater productivity, higher quality fruit, and earlier production; it is similar to ‘Ventana’ (U.S. Plant Pat. No. 13,469) and ‘Benicia’ (U.S. Plant Pat. No. 22,542), but with somewhat later production, a larger plant, superior fruit and quality, and better-flavored fruit.

### BRIEF DESCRIPTION OF THE DRAWINGS

The Figures depict various characteristics of the ‘Fronteras’ cultivar.

FIG. 1 shows the general flowering and fruiting characteristics of the plant in a field planting.

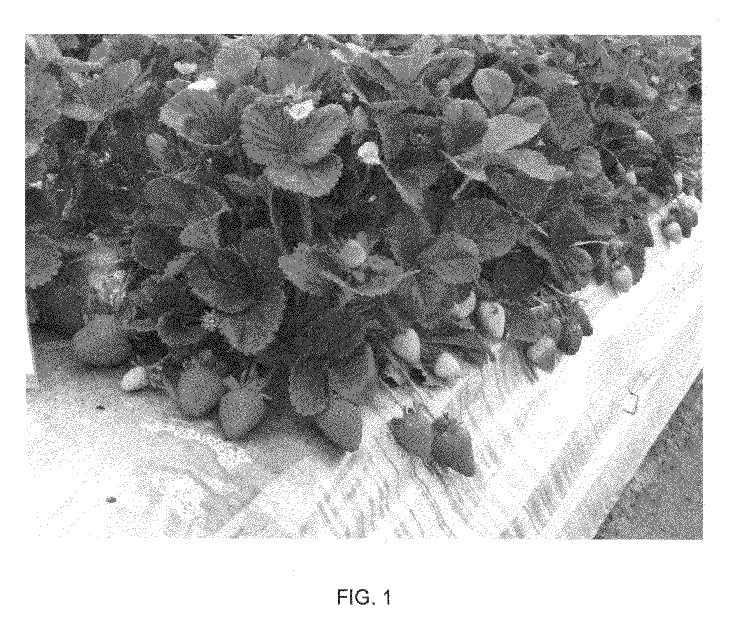

FIG. 2 shows a typical leaf at mid-season.

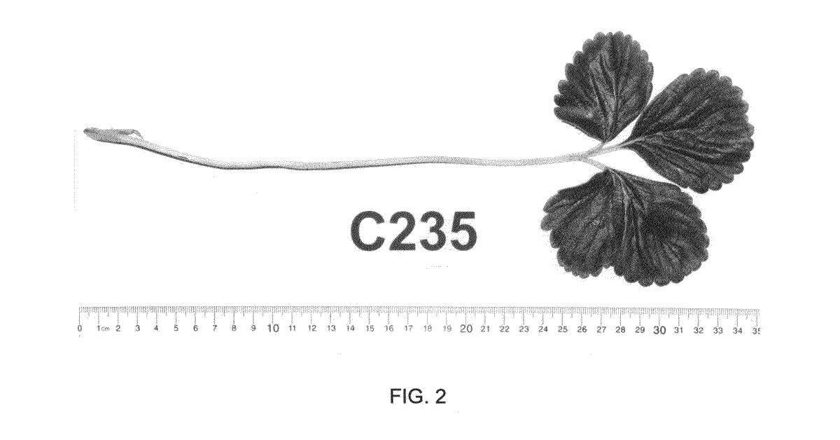

FIG. 3 shows representative mid-season fruit.

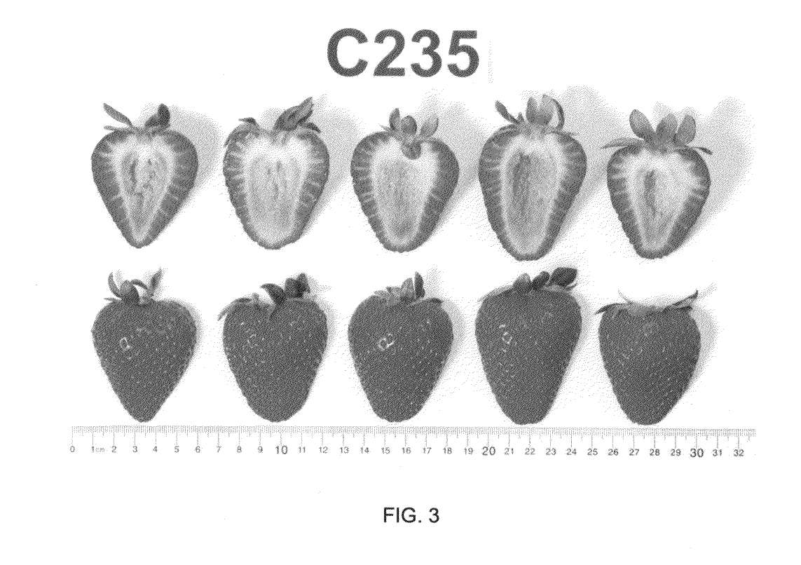

### DETAILED DESCRIPTION OF THE INVENTION

‘Fronteras’ is typical of short-day strawberry cultivars and produces fruit over an extended period when treated appropriately in arid, subtropical climates. The production pattern for ‘Fronteras’ is similar to that for ‘Camarosa’ (U.S. Plant Pat. No. 8,708), although it is slightly earlier to initiate fruiting with most cultural treatments. ‘Fronteras’ initiates fruiting slightly later than

‘Ventana’ (U.S. Plant Pat. No. 13,469) and ‘Benicia’ (U.S. Plant Pat. No. 22,542) when established in very early fall. ‘Fronteras’ will be of special interest for winter plantings, where ‘Camarosa’, ‘Ventana’, and ‘Benicia’ have been successful, and in summer plantings where ‘Chandler’ (U.S. Plant Pat. No. 5,262) and ‘Camino Real’ (U.S. Plant Pat. No. 13,079) have been successful.

### Plants and Foliage:

Fruiting plants of ‘Fronteras’ are slightly taller, more erect, and more open than all of the comparison cultivars in most production environments. Comparative statistics for foliar characters near mid-season are given for ‘Fronteras’ and three comparison cultivars in Table 1. Individual leaflets for ‘Fronteras’ are to those of the comparison cultivars, and are more elongated than for ‘Benicia’. Leaves (including petioles) for ‘Fronteras’ are slightly longer than for

‘Ventana’ and ‘Camarosa’, shorter than for ‘Benicia’. Petioles for ‘Fronteras’ are generally longer than those of ‘Ventana’, ‘Benicia’ and ‘Camarosa’. The adaxial (upper) and abaxial (lower) surfaces of leaves for ‘Fronteras’ are lighter than for ‘Camarosa’ and ‘Benicia’, darker and less yellow than for ‘Ventana’ leaves at midseason. Leaves of ‘Fronteras’ have similar concavity to ‘Camarosa’, and are less concave those for ‘Ventana’. Serrations at midseason are less pointed than for ‘Ventana’, similar in shape and number to ‘Benicia’ and ‘Camarosa’. The stipule length is somewhat longer for ‘Fronteras’ than for the comparison cultivars.

TABLE 1
Foliar and plant characteristics for ‘Fronteras’, ‘Camarosa’, ‘Ventana’, and ‘Benicia’.

| | Cultivar | | | |
|---|---|---|---|---|
| Foliar Character | ‘Camarosa’ | ‘Ventana’ | ‘Benicia’ | ‘Fronteras’ |
| Plant height (mm) | | | | |
| mean | 227 | 277 | 245 | 313 |
| range | 190-320 | 250-300 | 220-260 | 300-330 |
| Plant spread (mm) | | | | |
| mean | 368 | 425 | 414 | 421 |
| range | 300-465 | 375-525 | 360-500 | 345-485 |
| Mid-tier leaflet | | | | |
| Length (mm) | | | | |
| mean | 85 | 89 | 80 | 83 |
| range | 70-95 | 80-110 | 70-90 | 80-90 |
| Width (mm) | | | | |
| mean | 79 | 77 | 80 | 73 |
| range | 65-90 | 70-90 | 75-80 | 60-90 |
| Mid-tier leaf | | | | |
| Length (mm) | | | | |
| mean | 230 | 231 | 264 | 247 |
| range | 200-290 | 180-260 | 220-310 | 200-280 |
| Width (mm) | | | | |
| mean | 143 | 153 | 161 | 141 |
| range | 120-170 | 140-160 | 150-180 | 120-160 |
| Leaf components | | | | |
| Petiole length (mm) | | | | |
| mean | 110 | 113 | 136 | 141 |
| range | 90-150 | 80-120 | 110-160 | 110-160 |
| Petiole diameter (mm) | | | | |
| mean | 3.6 | 5.3 | 4.9 | 4.6 |
| range | 3-4 | 4-7 | 4-6 | 4-5 |
| Petiolule length (mm) | | | | |
| mean | 5.1 | 6.9 | 5.3 | 5.7 |
| range | 4-6 | 6-8 | 4-6 | 4-7 |
| #leaflets/leaf | 3 | 3 | 3, rarely 4 or | 3 |
| | | | 5 | |
| Leaf convexity | most flat to | flat to very | flat to | flat to |
| | slight concave | concave | concave | concave |
| Serrations | | | | |
| number/leaf | 20.8 | 20.6 | 20.5 | 20.1 |
| range | 19-23 | 18-25 | 18-23 | 18-22 |
| shape | semi-pointed | semi-pointed | round to | round to semi- |
| | | | semi-pointed | pointed |
| Leaf pubescence | light- | moderate- | moderate- | moderate |
| | moderate | heavy | light | |
| Petiole pubescence | | | | |
| density | heavy | moderate- | heavy | moderate- |
| | | heavy | | heavy |
| direction | perpendicular | perpendicular | perpendicular | perpendicular |
| | | to acropetal | | to acropetal |
| Petiole color (Munsell) | 2.5 GY 8/9 | 7.5 GY 9/4 | 7.5 GY 8/10 | 2.5 GY 7/10 |
| Stipule length (mm) | | | | |
| mean | 27.2 | 24.0 | 31.1 | 37.5 |
| range | 20-34 | 20-30 | 25-40 | 30-40 |
| Stipule color | | | | |
| core | 2.5 Y 6/8 | 2.5 GY 8/9 | 2.5 Y 9/4 | 7.5 GY 8/7 |
| margms | 7.5 Y 6/7 | 5 GY 8/8 | 5 GY 8/8 | 5 GY 8/8 |
| Stolon base diameter (mm) | 11.7 | 15.2 | 16.5 | 13.2 |
| Stolons per nursery mother | 22.7 | 18.8 | 22.9 | 23.0 |
| plant | | | | |
| Venation | | | | |
| pattern | pinnate | pinnate | pinnate | pinnate |
| color | 7.5 GY 8/7 | 7.5 GY 9/4 | 7.5 GY 8/7 | 2.5 GY 9/8 |

### Disease and Pest Reaction:

‘Fronteras’ is moderately resistant to powdery mildew (Sphaerotheca macularis), moderately susceptible to Anthracnose crown rot (Colletotrichum acutatum), and moderately resistant to Verticillium wilt (Verticillium dahliae), Phytophthora crown rot (Phytophthora cactorum) and common leaf spot (Ramularia tulasnei) (Table 2). When treated properly, it has tolerance to two-spotted spider mites (Tetranychus urticae) equal to that for the comparison cultivars. ‘Fronteras’ is tolerant to strawberry viruses encountered in California.

TABLE 2
Disease resistance scores for ′Fronteras′ and three comparison cultivars;
all scores were obtained in evaluations conducted in 2012-2013.

| | Phytophthora | Verticillium | Colletotrichum |
|---|---|---|---|
| | Resistance Score | Resistance Score | Resistance Score |
| Genotype | (5 = best) | (5 = best) | (5 = best) |
| ′Cam arosa′ | 3.6 | 2.8 | 2.3 |
| ′Ventana′ | 2.1 | 2.9 | 3.0 |
| ′Benicia′ | 3.5 | 1.6 | 2.5 |
| ′Fronteras′ | 4.1 | 3.7 | 2.5 |

### Flowering, Fruiting, Fruit, and Production Characteristics:

‘Fronteras’ is similar to other California short-day strawberry cultivars (e. g. ‘Ventana’, ‘Camarosa’, and ‘Benicia’) in that it will flower over an extended period and into spring or summer, given appropriate local temperature and horticultural conditions. With most planting treatments ‘Fronteras’ produces fruit slightly later than ‘Ventana’ and ‘Benicia’ and earlier than for

‘Camarosa’. Comparative statistics for flower and fruit characters near mid-season are given for the four cultivars in Table 4. The primary flowers for ‘Fronteras’ are slightly larger than for ‘Camarosa’ but smaller than ‘Ventana’ and ‘Benicia’ with a calyx that is distinctly larger than the corolla on primary fruit. The calyx for ‘Fronteras’ varies in position but frequently has a slight indent early in the season and is even with the fruit later in the season; each primary flower has 5-7 petals, similar to the comparison cultivars on average. The fruit shape for ‘Fronteras’ is consistent throughout the season, and is typically medium to long conic, with a tendency to be somewhat cylindrical and blunt. It is easily distinguished by fruit shape from ‘Camarosa’ (shortened and flattened conic), or ‘Ventana’ (medium symmetrical conic), and ‘Benicia’ (often flattened). Fruit size for ‘Fronteras’ is substantially larger than for the comparison cultivars.

External fruit color for ‘Fronteras’ is similar to that for ‘Camarosa’, lighter than for ‘Benicia’, and darker than for ‘Ventana’; internal color for ‘Fronteras’ is somewhat lighter than for the comparison cultivars (Table 3). Achenes vary from yellow to dark red, and are even with the fruit surface or slightly indented.

TABLE 3
Foliar and fruit color characteristics for
′Fronteras′ and three comparison cultivars.

| | Cultivar | | | |
|---|---|---|---|---|
| Color Character | ′Camarosa′ | ′Ventana′ | ′Benicia′ | ′Fronteras′ |
| Leaf color | | | | |
| (CIELAB) | | | | |
| Adaxial | | | | |
| L* | | | | |
| mean | 38.3 | 39.2 | 35.0 | 38.3 |
| range | 37.3-39.8 | 36.0-41.1 | 33.3-36.4 | 34.8-41.1 |
| a* | | | | |
| mean | −12.2 | −14.3 | −11.7 | −13.0 |
| range | −9.5-−15.5 | −12.9-−16.7 | −10.3-−13.5 | −11.3-−15.6 |
| b* | | | | |
| mean | 16.9 | 20.6 | 16.9 | 18.7 |
| range | 13.3-19.9 | 17.3-24.8 | 13.1-21.7 | 13.8-22.6 |
| Munsell | 5 GY 5/5 | 2.5 GY 6/8 | 5 GY 5/6 | 5 GY 4/3 |
| Abaxial | | | | |
| L* | | | | |
| mean | 52.5 | 53.2 | 48.5 | 48.9 |
| range | 51.3-54.6 | 51.8-54.6 | 41.7-52.3 | 40.2-51.2 |
| a* | | | | |
| mean | −13.1 | −14.2 | −13.5 | −14.1 |
| range | −11.4-−14.9 | −13.9-−14.7 | −11.9-−16.8 | −13.0-−15.1 |
| b* | | | | |
| mean | 20.5 | 21.7 | 20.0 | 21.4 |
| range | 18.9-22.4 | 20.3-23.3 | 17.9-21.9 | 20.0-21.9 |
| Munsell | 7.5 GY 8/7 | 10 GY 8/7 | 7.5 GY 5/7 | 10 GY 7/8 |
| Fruit color | | | | |
| (CIELAB) | | | | |
| External | | | | |
| L* | | | | |
| mean | 38.6 | 38.1 | 36.0 | 36.9 |
| range | 34.7-42.7 | 37.6-39.0 | 34.2-37.5 | 35.5-37.3 |
| a* | | | | |
| mean | 34.4 | 33.4 | 31.2 | 37.3 |
| range | 33.6-36.2 | 29.4-38.7 | 26.6-36.3 | 35.1-39.9 |
| b* | | | | |
| mean | 22.5 | 19.2 | 14.2 | 19.2 |
| range | 18.8-29.3 | 17.8-21.1 | 10.6-17.3 | 16.7-19.0 |
| Munsell | 7.5 R 4/11 | 5 R 4/12 | 2.5 R 4/0 | 7.5 R 4/11 |
| Internal | | | | |
| L* | | | | |
| mean | 50.2 | 48.6 | 44.0 | 55.7 |
| range | 46.6-53.3 | 46.2-52.3 | 40.8-47.0 | 50.4-60.4 |
| a* | | | | |
| mean | 30.8 | 28.9 | 30.9 | 20.9 |
| range | 25.6-35.4 | 23.5-33.0 | 27.8-33.6 | 18.1-25.9 |
| b* | | | | |
| mean | 30.1 | 31.3 | 27.5 | 25.4 |
| range | 28.0-32.0 | 30.6-32.5 | 24.6-28.8 | 19.6-30.7 |
| Munsell | 7.5 R 5/13 | 7.5 R 6/13 | 5 R 4/2 | 7.5 R 5/3 |
| Achene color | 2.5 Y 7/10 | 10 Y 8/11 | 5 R 3/7 | 2.5 R 8/12 |
| Munsell | | | | |

TABLE 4
Flower and fruit characters for ′Fronteras′ and three comparison cultivars.

| | Cultivar | | | |
|---|---|---|---|---|
| Character | ′Camarosa′ | ′Ventana′ | ′Benicia′ | ′Fronteras′ |
| Petal number | | | | |
| mean | 5.8 | 6.2 | 6.1 | 5.9 |
| range | 5-7 | 5-7 | 5-7 | 5-7 |
| Petal shape | | | | |
| apex | truncate to | truncate to | truncate to | truncate to |
| slightly | slightly | slightly | slightly | |
| base | obtuse | obtuse | obtuse | obtuse |
| margin | attenuate | attenuate | attenuate | attenuate |
| entire | entire | entire | entire | |
| Petal length (mm) | | | | |
| mean | 11.5 | 13.3 | 11.7 | 13.5 |
| range | 10-13 | 11-15 | 8-13 | 13-15 |
| Petal width (mm) | | | | |
| mean | 12.0 | 14.6 | 14.4 | 12.6 |
| range | 10-14 | 13-16 | 8-13 | 8-14 |
| Flower position | most even | even to | even to | most even |
| (relative to foliage) | some | exposed | exposed | some |
| exposed | interior | | | |
| Calyx diam.(mm) | | | | |
| mean | 40.4 | 47.0 | 50.8 | 48.3 |
| range | 33-47 | 40-50 | 47-53 | 44-54 |
| Corolla diam.(mm) | | | | |
| mean | 26.1 | 39.0 | 39.6 | 31.3 |
| range | 23-31 | 35-45 | 39-41 | 29-38 |
| Sepal length (mm) | | | | |
| mean | 14.3 | 16.6 | 16.4 | 14.6 |
| range | 12-18 | 14-19 | 13-20 | 11-17 |
| Sepal width (mm) | | | | |
| mean | 8.3 | 8.4 | 8.4 | 9.3 |
| range | 7-10 | 7-10 | 7-10 | 7-11 |
| Sepal color (Munsell) | 5 GY 7/10 | 5 GY 5/5 | 10 GY 8/7 | 5 GY 5/6 |
| Pedicel length (mm) | | | | |
| mean | 155 | 115 | 183 | 125 |
| range | 130-180 | 90-140 | 150-210 | 90-170 |
| Pedicel diameter (mm) | | | | |
| mean | 2.7 | 3.5 | 3.7 | 4.7 |
| range | 2-4 | 3-4 | 3-5 | 4-6 |
| Pedicel color | 7.5 GY 8/7 | 5 GY 8/9 | 2.5 GY 8/9 | 7.5 GY 6/8 |
| Fruit shape | | | | |
| Fruit length (mm) | | | | |
| mean | 46.0 | 48.4 | 46.5 | 54.5 |
| range | 40-48 | 47-52 | 41-52 | 51-58 |
| Fruit width (mm) | | | | |
| mean | 37.4 | 42.6 | 42.4 | 46.7 |
| range | 33-46 | 40-46 | 36-46 | 42-54 |
| Length/ width | | | | |
| ratio | 1.26 | 1.17 | 1.08 | 1.15 |
| range | 1.0-1.4 | 1.1-1.2 | 1.0-1.2 | 1.0-1.2 |
| subjective | Obovate-flat | Medium | Medium | Medium-long |
| conic | conic | conic | | |
| Primary/secondary fruit | | | | |
| comparison | | | | |
| size (subjective) | 50-70% | 55-75% | 55-65% | 60-80% |
| shape | similar | similar shape | similar shape | similar shape |
| | shape, more | | | |
| | conic | | | |
| Extent/size of hollow core | small-absent | small | small-absent | small-absent |
| Calyx position | indented- | indent- | even-indented | Indented-even |
| | neck | reflexed | | |
| size relative to fruit | equal or less | equal or less | equal or | equal or less |
| | than fruit | than fruit | greater than | than fruit |
| | diameter | diameter | fruit diameter | diameter |
| Seed position | indented- | mostly even | even-indented | indented- |
| | extruded | | | extruded |
| Adherence of Calyx to Fruit | weak | intermediate | weak | intermediate |

Flower and plant measurements obtained on April, 2012, fruit measurements May 10-20, 2012.

‘Fronteras’ has been tested under a variety of cultural regimes, and optimal performance is obtained when nursery treatments and nutritional programs similar to those for ‘Camarosa’, ‘Ventana’, and ‘Benicia’ are used. In general, plants of ‘Fronteras’ are greater in vigor than the comparison cultivars with very early season planting. ‘Fronteras’ retains excellent fruit quality in summer planting systems.

When treated with appropriate planting regimes, ‘Fronteras’ has substantially larger sized fruit and produces individual-plant yields greater than any of the comparison cultivars (Table 5). Commercial appearance ratings have also been substantially better than those for all of the comparison cultivars, especially in comparison with ‘Camarosa’. Fruit for ‘Fronteras’ is similar in firmness to fruit from ‘Ventana’, less firm than the other comparison cultivars. Subjectively, ‘Fronteras’ has excellent flavor. The fruit will be exceptional for both fresh market and processing, and will be useful for home garden purposes.

TABLE 5
′Fronteras′ and three comparison cultivars evaluated at the Watsonville Research
Facility in 2010-12. All plants for these trials were harvested from a commercial nursery near
Macdoel, CA on October 15-16, and transplanted after 6-7 days supplemental storage. Fruit
harvest was initiated in early April and continued through the last week of August. (52″ 2-row
beds, 17,300 plants/acre).

| | | Appearance | Fruit | |
|---|---|---|---|---|
| | Yield | Score | Size | |
| Item | (g/plant) | (5 = best) | (g/fruit) | Firmness |
| ′Camarosa′ | 1,815 | 2.8 | 27.1 | 11.6 |
| ′Ventana′ | 2,080 | 3.3 | 30.1 | 10.2 |
| ′Benicia′ | 1,649 | 3.4 | 33.1 | 11.1 |
| ′Fronteras′ | 2,793 | 4.2 | 35.1 | 11.1 |

What is claimed is:

1. A new and distinct cultivar of strawberry plant having the characteristics substantially as described and illustrated herein.
